(19) United States
(12) Patent Application Publication
(10) Pub. No.: US 2017/0282822 A1
(43) Pub. Date: Oct. 5, 2017

(54) CHILD VEHICULAR ABANDONMENT PREVENTION SYSTEM

(71) Applicant: CECIL LEE HUNTER, JR., Apopka, FL (US)

(72) Inventor: CECIL LEE HUNTER, JR., Apopka, FL (US)

(21) Appl. No.: 15/469,354

(22) Filed: Mar. 24, 2017

Related U.S. Application Data
(60) Provisional application No. 62/317,587, filed on Apr. 3, 2016.
(60) Provisional application No. 62/348,964, filed on Jun. 12, 2016.

(51) Int. Cl. B60R 16/037; B60R 22/10; B60R 22/48; B60K 35/00; B60K 15/03; B60N 2/00; B60N 2/26; B60Q 5/00; E05B 81/54; B60H 1/00; A61B 5/01; A01K 11/00; A01K 29/00; A61B 5/00; H04L 29/08; G08B 21/02; B60R 16/02

(52) U.S. Cl. CPC B60R 16/037; B60R 16/0207; B60R 22/105; B60R 22/48; B60K 35/00; B60K 15/03; B60N 2/002; B60N 2/26; B60Q 5/005; E05B 81/54; B60H 1/00978; A61B 5/01; A01K 11/006; A01K 29/005; A61B 5/742; H04L 67/12; G08B 21/02; B60R 2022/4816; B60K 2350/106; B60K 2350/1096; B60K 2350/965; B60K 2015/0323

(57) ABSTRACT

The present invention, in one form, is an absolute prevention system that will not allow anyone to leave an infant/child or pet in a vehicle unattended mistakenly or intentionally. The system comprises an ECM/ECU with auxiliary wiring connections from the host vehicle wiring, such as the ignition, A/C and heat, door ajar, vehicle seat weight sensors and ABS sensor, interior display screen, door locks, power windows, interior speakers and the vehicles' theft alarm system. In addition, the ECM/ECU will allow connections for a flow sensor/meter, passive infrared sensors, temperature sensor, wireless antenna, voice module, ISO pet chip reader and a hardwired harness. The ECM/ECU is capable of employing 911, GPS location and text message logged love ones.

### CROSS REFERENCE TO RELATED APPLICATION

The present application claims priority to U.S. provisional application Ser. No. 62/317,587 filed Apr. 3, 2016 and U.S. provisional application Ser. No. 62/348,964 filed Jun. 12, 2016. The contents of which are incorporated herein.

### BACKGROUND OF THE INVENTION

Since 1998, an average of 37 kids per year (one every 9 days) have died of heat stroke due to being left in vehicles with the highest number of fatalities for a one-year time period being in 2010 with 49 deaths. From 1990-2015, Child vehicular heat stroke deaths total 755. It is easy to place a child in a vehicle and head off to work via the daycare but forget a child is onboard if the infant/child falls asleep during the commute; the driver is on their cell phone or emerge themselves into their favorite radio broadcast.

Throughout the course of researching the referenced cited patents, the search revealed several of them that use temp sensors as the source to provoke the system to alarm. Temperature is the cause of heat stroke deaths so why would an invention that seek to end heat stroke deaths flirt with temperature.

Some of these inventions do not incorporate a driver exit alert and allows a driver to leave the vehicle with the child left inside. Those that do offer an exit alert suggest after a certain amount of time has lapsed and the temperature had reached a certain threshold, the vehicle alarm system would be triggered to alert of the situation.

The patent search also revealed inventions that if a child has been left inside a vehicle, not only will it alarm when temperature reach a certain point but by motion as well. Understand that a sleeping child maybe motionless so now these systems will wait for temperature to reach a set threshold.

The search revealed inventions that have removable sensors that in the long run could possibly be damaged or lost and inventions that allows the driver to turn the systems off.

The search found that some of the cited inventions don't incorporate continual monitoring throughout driving range and car seats with receiver units adjacent to the vehicle belt buckle on the vehicle seat oriented towards a transmission arrangement so that it could send and receive alerts. If this receiver unit is out of alignment or blocked, the driver wouldn't get any alerts.

The search found inventions that use different colored flashing lights for alerting the driver of issues. Inventions that have mobile controllers which allows the caregiver who has the controller to LEAVE OCCUPANTS UNATTENDED in a vehicle but is warned via the controller of any threats inside the vehicle in which the same invention have hardware/cladding for self-contained independent sensor monitoring modules that are deployed on the designated individuals and pets to be monitored with a master control communication and command module unit carried by the designated user to monitor and maintain the safety of the monitored entities from a distance.

The cited patent search found an invention that suggest “the alarm is activated when the car key is removed from the ignition and the baby is still buckled in the car seat”. This somewhat suggest that someone could leave the key in the ignition and exit the vehicle with the child still in the vehicle. The system has no deterrent for the caregiver, it just alerts the caregiver of the child's presence but has gaps that a caregiver could exploit.

The cited patent search found inventions that will turn on A/C but suggest no way of determining if the A/C or heat is needed in a possible abandonment situation of a child.

The cited patent search found inventions that suggest lowering the window of a vehicle to make it comfortable for an abandon child and unlocking the doors while using the vehicle alarm to attract attention to inside of the vehicle. Alarming vehicles don't arouse people attention to look inside of them while unlocking the doors now leaves the child without security and toweling the windows will leave a child vulnerable to the elements.

Of all the referenced cited patents, when alarming if a child is left in a vehicle, NONE offered verbal exterior voice module alerting the public to the LIFE-THREATENING SITUATION INSIDE OF THE VEHICLE. They all state the use of the vehicle alarm system which is the horn blowing and vehicle lights flashing to alert the public of a child left inside the vehicle. Alarming vehicles don't spark people interest to look inside them, so there's a high probability that the child wouldn't be rescued.

A number of the cited patents suggest allowing a caregiver/owner the ability to turn off the system, lull or adjust its parameters by adding time to give themselves more time before alarming wherein the Child Vehicular Abandonment Prevention System only allows a child to be unsupervised for 4 minutes before it is triggered to alarm.

None offered a refueling mode that continues the accountability of protecting a child from abandonment or excessive length of time outside of the vehicle with a child still seated inside alone, A number cited patents suggest proximity sensors that will allow a caregiver to leave the vehicle for an undetermined amount of time if they stay within so many feet/yards of the vehicle.

None offered a systematic way of superintending the loading and unloading time of children and pets or the ability to monitor and see a sick child's temperature reading live over the vehicle display screen.

None have the capability to preserve the scene for investigation by locking the vehicle creating a secure environment and a comfortable interior atmosphere until a first responder arrive to unlock the vehicle and start an investigation.

The above described systems, however, tend to have flaws, gaps and lapse in their security features. It is the objective of the Child Vehicular Abandonment Prevention System to close all gaps and remove all flaws while creating a system with no lapse in security for a caregiver most precious cargo.

### BRIEF SUMMARY OF INVENTION

This Child Vehicular Abandonment Prevention System seeks to totally defunct heat stroke deaths and abandonment of children and pets in vehicles.

The said system embodies an ECM/ECU (Electronic Control Module/Electronic Control Unit) which will monitor, command and control the entire system. A number of vehicle functions such as the ABS speed sensors, interim′ display screen, vehicle interior speakers, door locks, power windows, the vehicles' alarm system, A/C and heat, ignition, door ajar and seat weight sensor will nexus to the ECM/ECU through an auxiliary wire harness which would be separate from the vehicles' wiring for the purpose of the lessor the integration the easier to diagnose problems that may arise and would be powered by vehicle electrical system.

Other constituents that will connect the ECM/ECU to assist in protecting a caregiver most precious cargo are, a flow (ultra) sensor/meter, temperature sensor, passive infrared sensors (P.I.R.), alarm voice module, an International Standards Organization (ISO) pet microchip reader, wireless antenna (wireless child seat connectivity), the capability to call 911 and text message logged love ones when secure mode is activated, a hardwired harness (bridges the ECM/ECU and Child seat via the rear redesigned seatbelt buckles) and redesigned seatbelt buckles that allow plug-in access to the hardwired harness for the child/infant seats and child/infant seats that continuously monitors throughout driving.

All the above embodiments when deployed in a vehicle as one integral infrastructure or any vessel capable of transporting children with the possibility of abandonment of a child or pet intentionally or mistakenly will be dissuade by this system.

### BRIEF DESCRIPTION OF DRAWINGS

The constituents set forth in the depictions are illustrative and paradigmatic in nature and not intended to limit the subject matter defined by the claims.

FIG. 1 depict a top view of the ECM/ECU with the hardwired harness connection connecting on the right side and the constituents connecting on the left side.

FIG. 1B depict an individual breakdown of ail the constituents that would connect to the ECM/ECU and its capabilities such as 911 call, text message logged love ones and wireless reception/transmission.

FIG. 2 depict a cut out view of a vehicle for optic of all the constituents and their positions and relationship to and in the vehicle.

FIG. 3 depict the position of a flow meter/sensor within the fuel filler neck.

FIG. 3B depict the position of an Ultra Sonic flow meter/sensor within the fuel filler neck.

FIG. 4 depict an exploded view of a ABS brake system showing the ECM/ECU connecting to the ABS control module for reading the ABS wheel speed sensors via the control module.

FIG. 5 depict a child secured in the child seat with the hardwire leading from the rear of the seat connecting into the access port of the redesigned seatbelt buckle; also applied to infant carriers.

FIG. 5B depict the child seat and the position of the ECM/ECU along with the hardwire leading from the ECM/ECU; also applied to infant carriers.

FIG. 5C depict the child seat with a weight/occupancy sensor covering the seating area and back support area and also displays the seatbelt sensor; also applied to infant carriers.

FIG. 6 depict the position and view of the temp sensor, ISO pet microchip reader, passive infrared sensors (P.I.R.) and the wireless antenna.

FIG. 7 depict the interior display screen displaying a logged childs' name, picture and child's temperature live onto the vehicle display screen.

FIG. 7A depict a logged child (name and photo) being welcomed into the vehicle and a happy birthday greeting once being placed into “their” child seat or infant carrier has been brought onboard.

FIG. 7B depict the interior display screen expressing the system is locked due to being in secure mood.

FIG. 7C depict the interior display screen displaying refuel access mode

FIG. 7D depict the interior display screen

FIG. 8 depict the redesigned seatbelt buckle and a hardwire access port for the child Seat/infant carrier hardwire to plug into.

FIG. 9 depict a key fob that is only capable of unlocking/disarming the system from secure mood.

FIG. B depicts the hardwire harness and it nexus points relative to the ECM/ECU and rear seatbelt buckles.

### DETAILED DESCRIPTION OF INVENTION

Every thought went into this system to ensure the safety of children and pets. Some of the systems that were searched have similar characteristics and components to this Child Vehicular Abandonment Prevention System but none come close nor offer the unimpeded protection, interfacing features or the potential to preserve the scene if child abandonment is pose while allowing authorities to investigate immediately. Through TempTraq (US 20140121557 A1) wearable, wireless, continuously monitoring intelligent thermometer, a child's temperature would be displayed live over the vehicle display screen offering a caregiver constant intelligence about their sick child. Said invention even incorporates possible circumventing deterrent and does not allow the system to be turned off, lulled or it's parameters adjusted. Said invention will alarm due to leaving a child unattended or possible abandonment, not by temperature threat. The most important of all is, the verbal acoustic ability to communicate the risk to the public that a child or pet has been left in a vehicle.

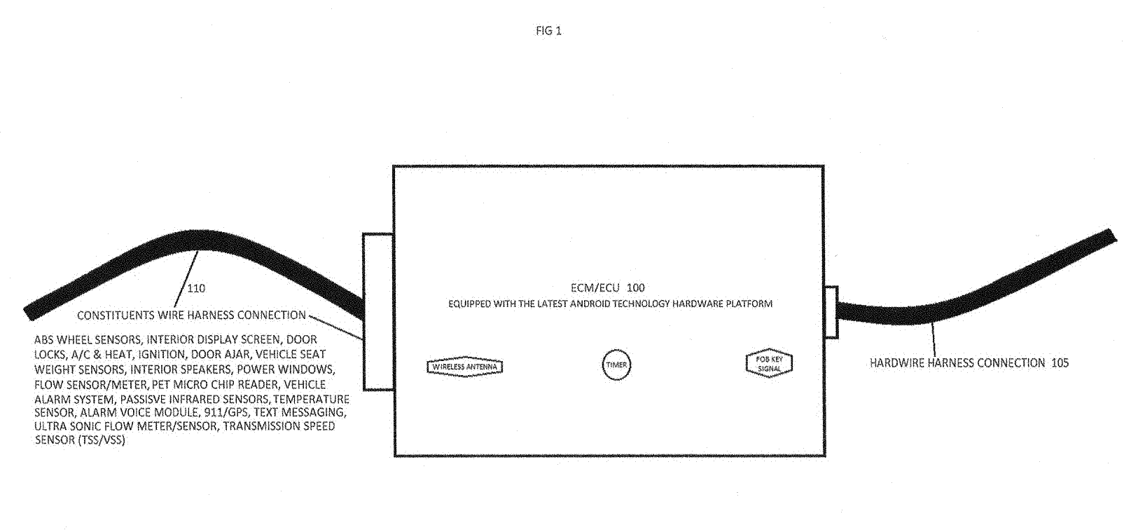

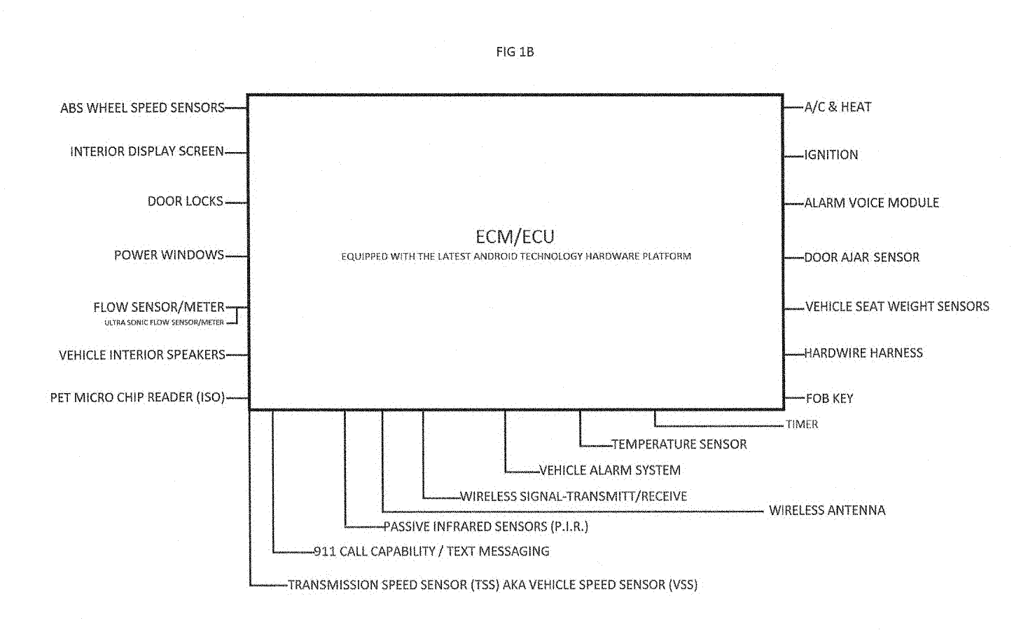

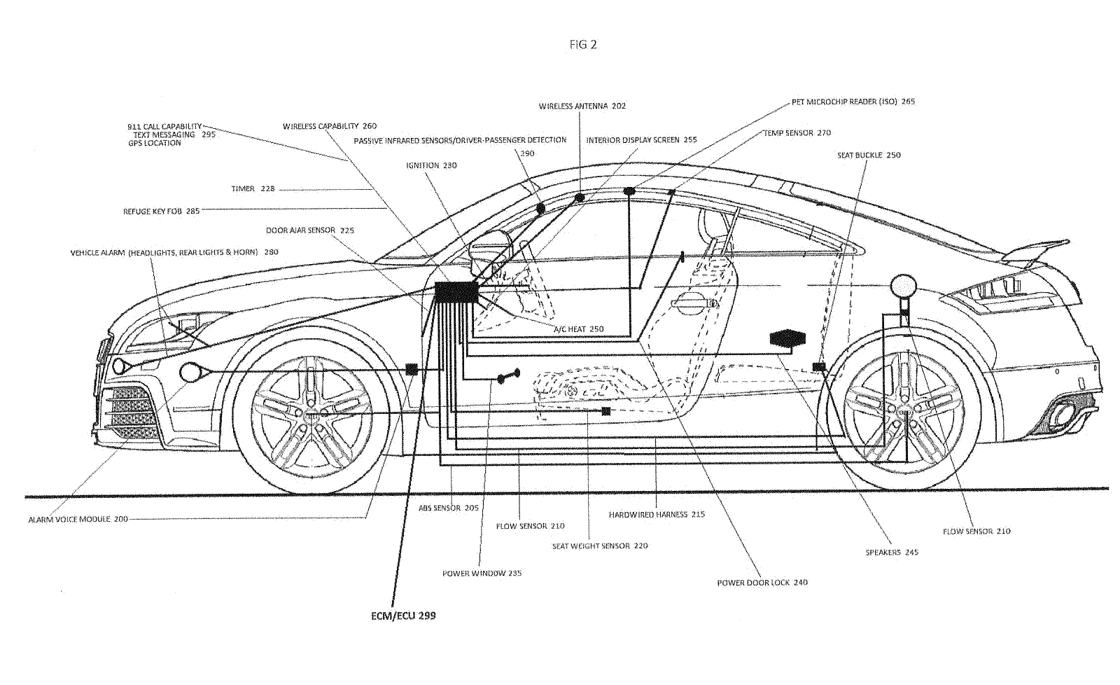

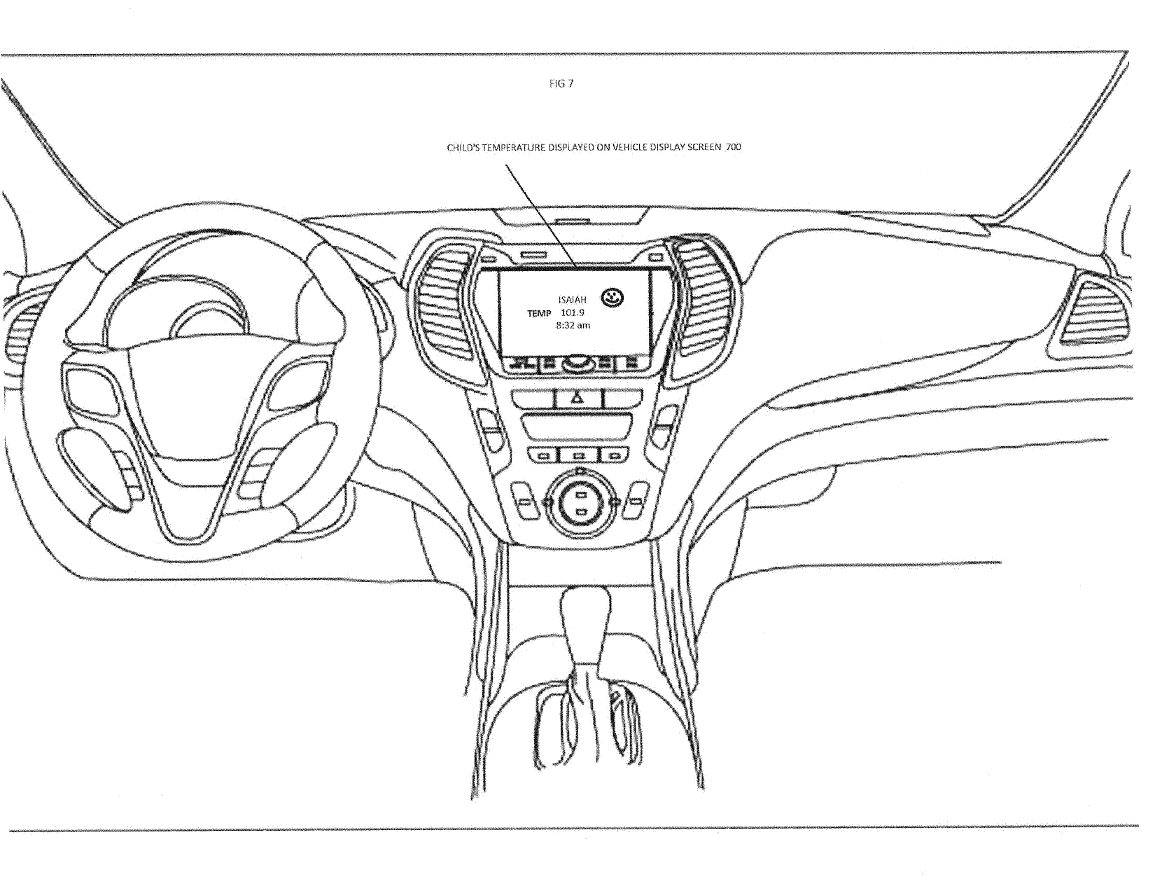

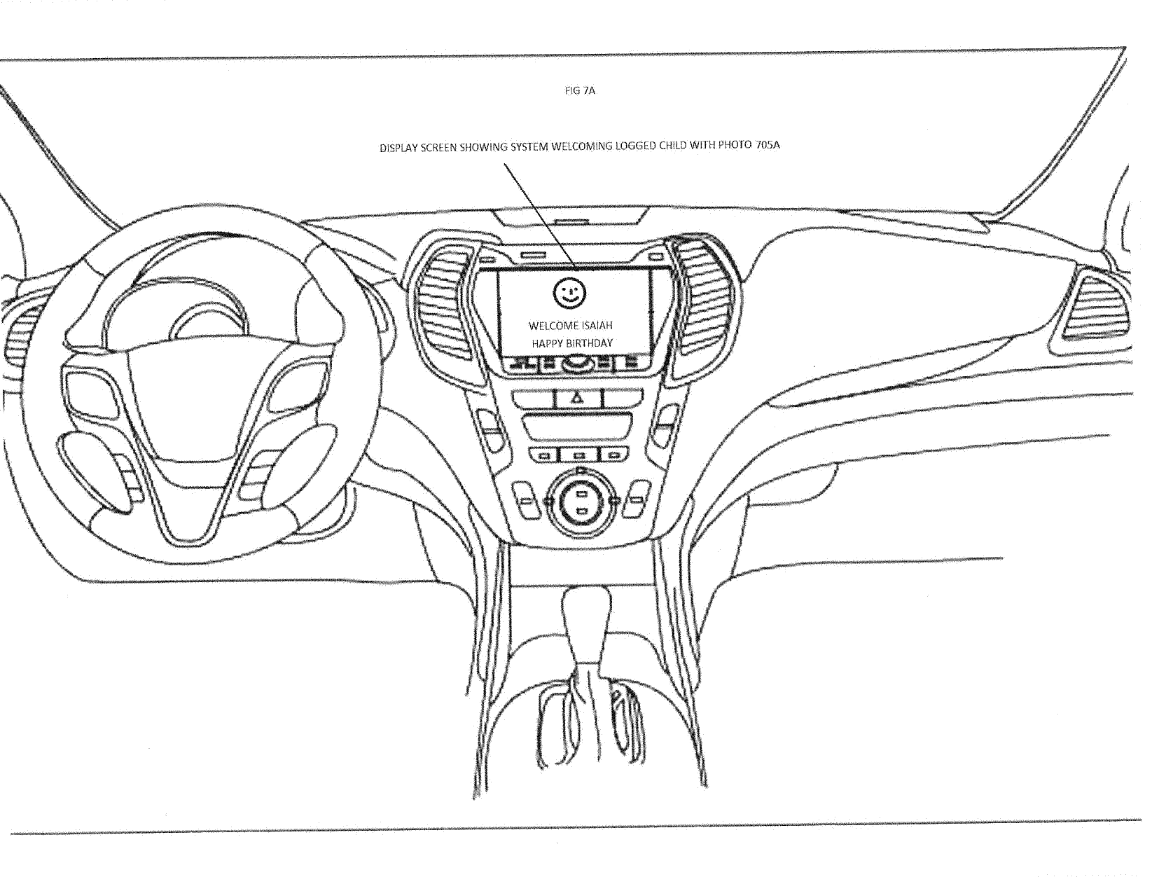

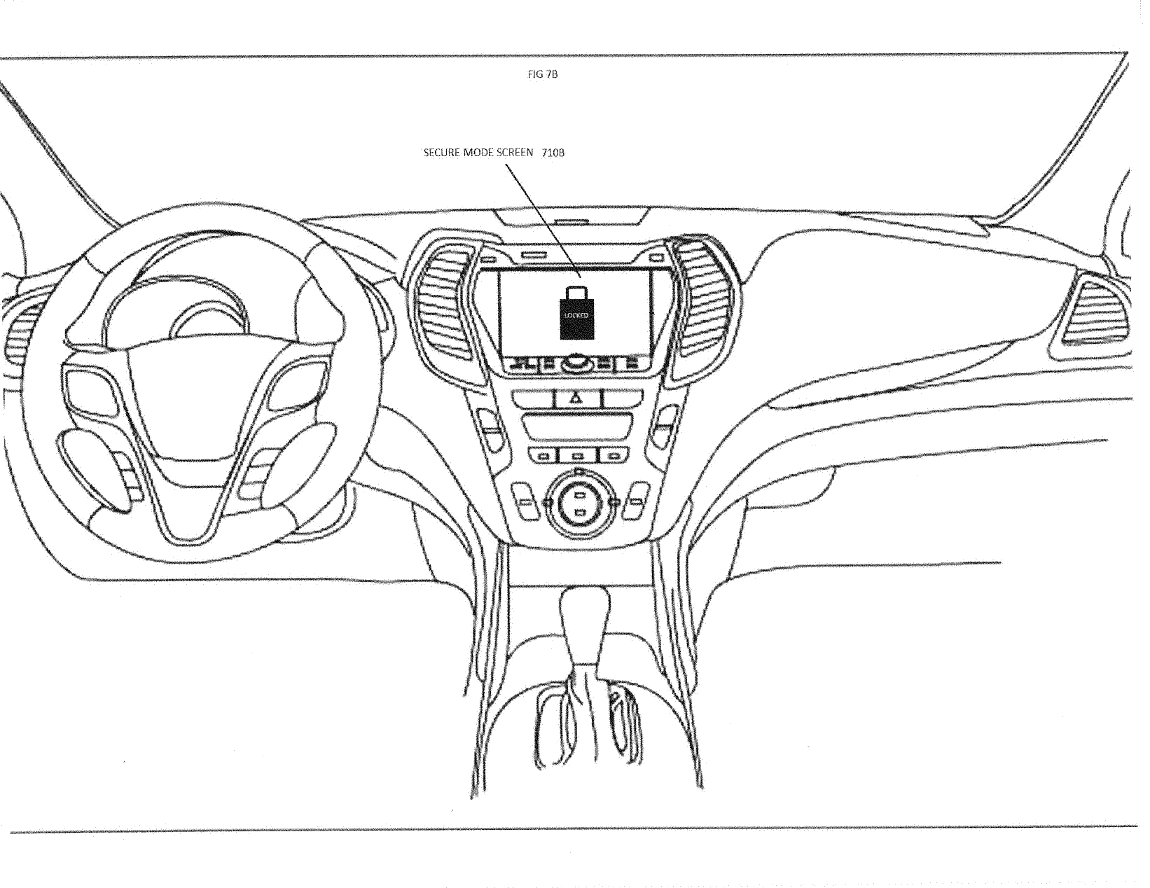

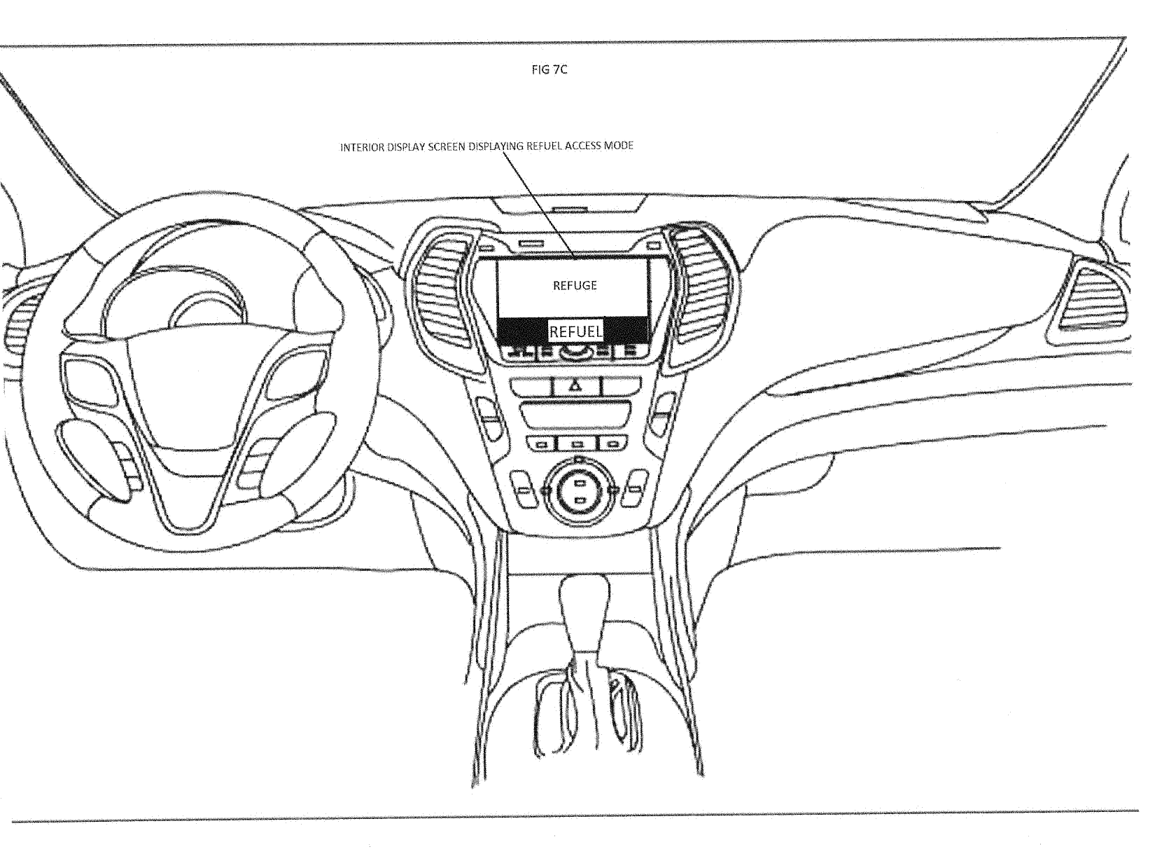

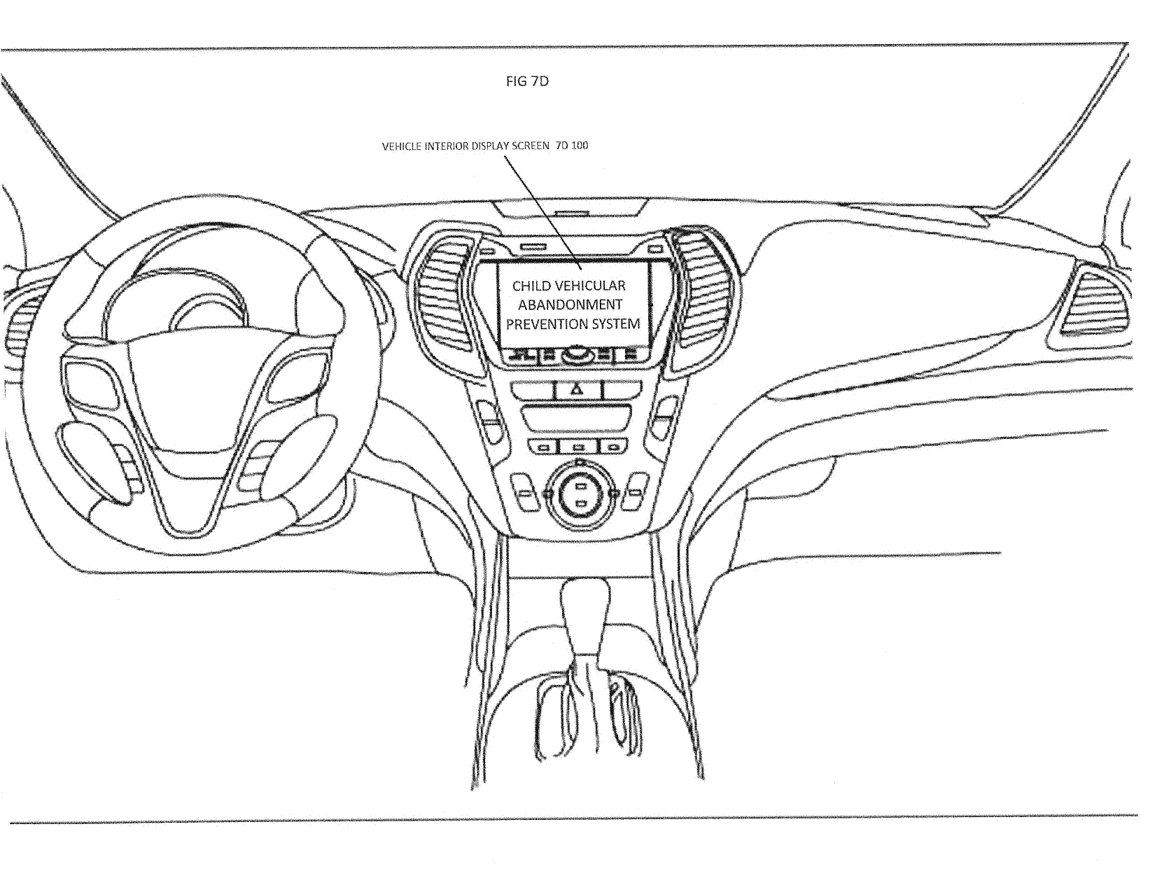

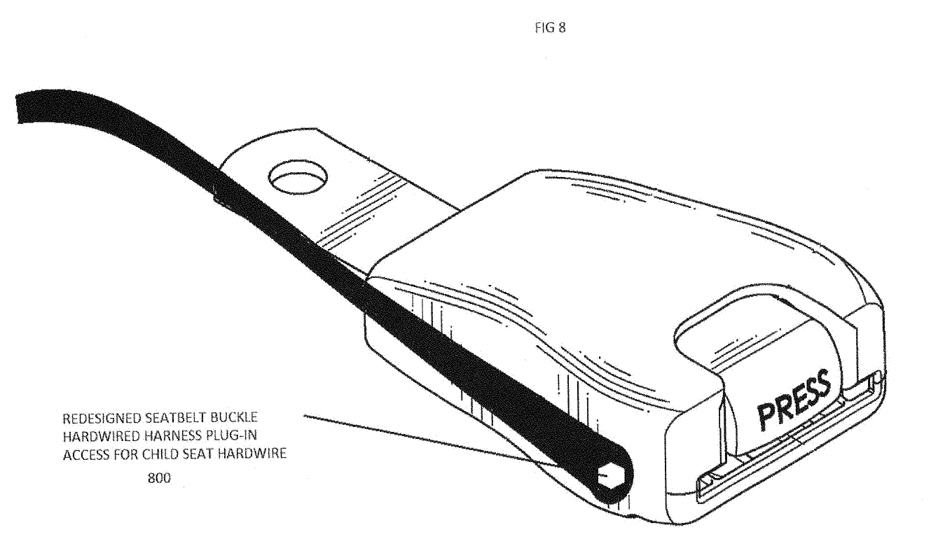

FIG. 1 depict a top view of the ECM/ECU FIGS. 1/100 with the hardwired harness connection FIGS. 1/105 on the right side and the constituents connecting FIGS. 1/110 on the left side. The ECM/ECU FIGS. 1, 1B & FIGS. 2/299 will be the controlling entity of the system. It is the electronic circuitry that carries out the instructions by performing the basic arithmetic, logical, control and input/output (I/O) operations specified by the instructions. It'll direct operation of the processor. It tells the computer's memory, arithmetic/logic unit and input/output (I/O) devices how to respond to a program's instructions. The vehicle auxiliary wiring would connect to it; it would not be wired into the vehicle wiring nor would the host vehicle computer control any functions of the system or ECM/ECU FIGS. 1, 1B & FIGS. 2/299. The lessor the integration, the easier it is to diagnose any issues that may arrive. The ECM/ECU FIGS. 1, 1B & FIGS. 2/299 will allow access to download events of alarming, for the reason of authoritative investigations. Equipped with the latest Android Technology hardware platform FIGS. 1/100 for interfacing and uploading information by vehicle owner such as names of kids and pets, pictures of kids and pets, birthdays or special events of kids and pets which would be displayed over the vehicle's display screen FIG. 7A and announced over the vehicle's speakers FIGS. 2/245 once the child is placed in their assigned seat or a pet comes on board allowing the ISO pet microchip reader FIGS. 2/265 to read the pets' chip. With wireless capability FIGS. 2/260 and Android Technology hardware platform, will allow the ECM/ECU FIGS. 1, 1B & FIGS. 2/299 to display the child's temp FIGS. 7/700 on the vehicle display screen FIGS. 7D/100 through TempTraq (US 20140121557 A1) wearable, wireless, continuously monitoring intelligent thermometer. The ECM/ECU FIGS. 1, 1B & FIGS. 2/299 will also allow upload of the caregiver and loved one's cell phone numbers for text message alert if system is triggered into secure mode FIGS. 2/200, 280, 295, 230, 240, 235, 250 & 270.

FIG. 1B depict an individual breakdown of all the constituents that would connect to the ECM/ECU FIGS. 1, 1B & FIGS. 2/299 and its capabilities such as 911 call/text message logged love ones FIGS. 2/295 and wireless reception/transmission FIGS. 2/260.

FIG. 2 depict a cut out view of a vehicle for optic of all constituents 200-299 and their positions and relationship to and in the vehicle. This allows the interpreter an autopsy view to the functional complexity.

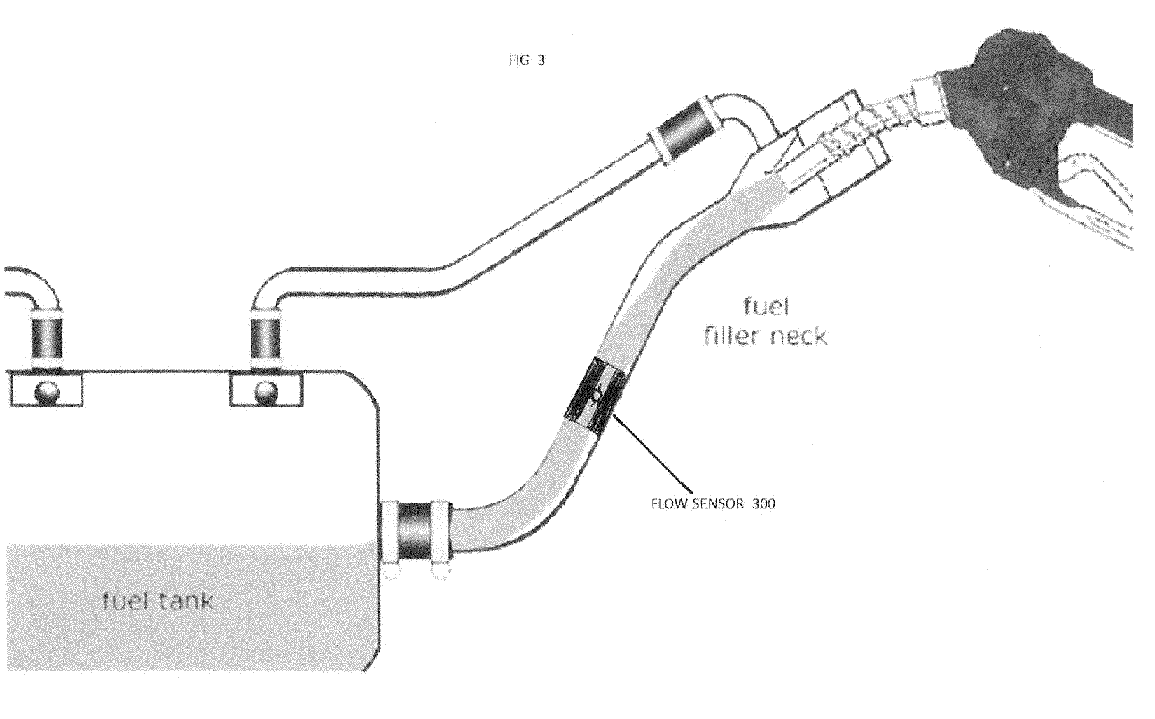

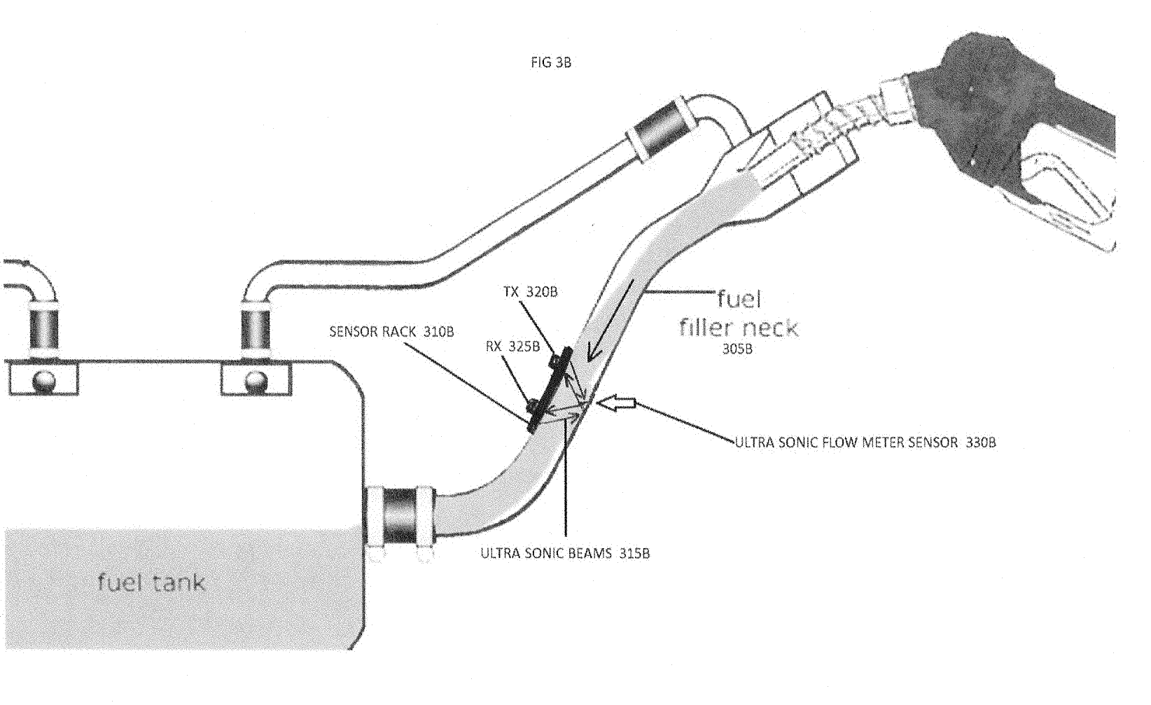

FIGS. 3 & 3B. FIG. 3 depict the position of a flow meter/sensor 300 within the fuel filler neck FIGS. 3B/305B & FIG. 3: FIG. 3B depict the position of an Ultra Sonic flow meter/sensor 330B within the fuel filler neck FIGS. 3B/305B. The flow meter sensor FIGS. 3/300 or FIGS. 3B/330B would be constructed in the fuel filler neck FIGS. 3B/305B of a vehicle to detect fuel flow. A vehicle could be equipped with either type. If a driver and child had to refuel, the driver would go to the vehicle display screen FIGS. 7D/100 and engage the refueling button FIG. 7C. It will allow the driver to exit the vehicle, removing weight from the seat weight sensor FIGS. 2/220 without the verbal interior message alarming over the vehicle interior speakers FIGS. 2/245 or the vehicle going into secure mode FIGS. 2/200, 280, 295, 230, 240, 235, 250 & 270. The ECM/ECU timer FIGS. 1/100 will allow 3 minutes to start fueling the vehicle. If fueling doesn't start within 3 minutes, the system would alarm (vehicle alarm only) FIGS. 2/280 for 1 minute before going into secure mode FIGS. 2/200, 280, 295, 230, 240, 235, 250 & 270 believing the child has been abandon. The 1 minute is to alert the driver and or that time has expired before going into full secure mode FIGS. 2/200, 280, 295, 230, 240, 236, 250 & 270 which in addition to the vehicle alarm, the alarm voice module, power door locks locking, ignition starting vehicle, elevating windows, temp sensor determining A/C or heat, NC or heat being turned on, text message a logged love one and dialing 911 and GPS location at 10 minutes of alarming. While alarming “during” the 1 minute vehicle system alarm FIGS. 2/280 before going into secure mode FIGS. 2/200, 280, 295, 230, 240, 235, 250 & 270, driver would still be able to start refueling thwarting system from going into secure triode FIGS. 2/200, 280, 295, 230, 240, 235, 250 & 270. Once the flow sensor FIGS. 3/300 & FIGS. 3B/305B detects flow, it would alert the ECM/ECU FIGS. 1, 1B & FIGS. 2/299 to cease the vehicle alarm FIGS. 2/280 and thwart the system from going into secure mode FIGS. 2/200, 280, 295, 230, 240, 235, 250 & 270. Once the driver has finished fueling and the flow sensor FIGS. 3/300 & 3B/305B doesn't detect flow, the flow sensor FIGS. 3/300 & FIGS. 3B/305B would signal the ECM/ECU FIGS. 1, 1B & FIGS. 2/299 to restart the 3-minute grace period to allow the caregiver to reoccupy the vehicle and sit in a weight sensor seat FIGS. 2/220 which would thwart alarming. If the caregiver doesn't reoccupy the vehicle before the 3 minutes expire, said invention would alarm (vehicle alarm only) FIGS. 2/280 for 1 minute before going into secure mode FIGS. 2/200, 280, 295, 230, 240, 235, 250 & 270 believing the child has been abandon. The 1 minute is to alert the driver and or that time has expired before going into full secure mode FIGS. 2/200, 280, 295, 230, 240, 235, 250 & 270 which in addition to the vehicle alarm, the alarm voice module, power door locks locking, ignition starting vehicle, elevating windows, temp sensor determining A/C or heat, NC or heat being turned on, text message a logged love one and dialing 911 and GPS location at 10 minutes of alarming. If a driver stops to refuel and there is a passenger in a weight sensor seat FIGS. 2/220, the driver wouldn't have to use refueling mode FIG. 7C. Refueling mode FIG. 7C is only for single occupant caregiver that would pay at the pump. If a driver chose to pay inside, they would have to remove the child or someone would have to be inside the vehicle occupying a weight sensor seat, the system cannot be turned off or lulled.

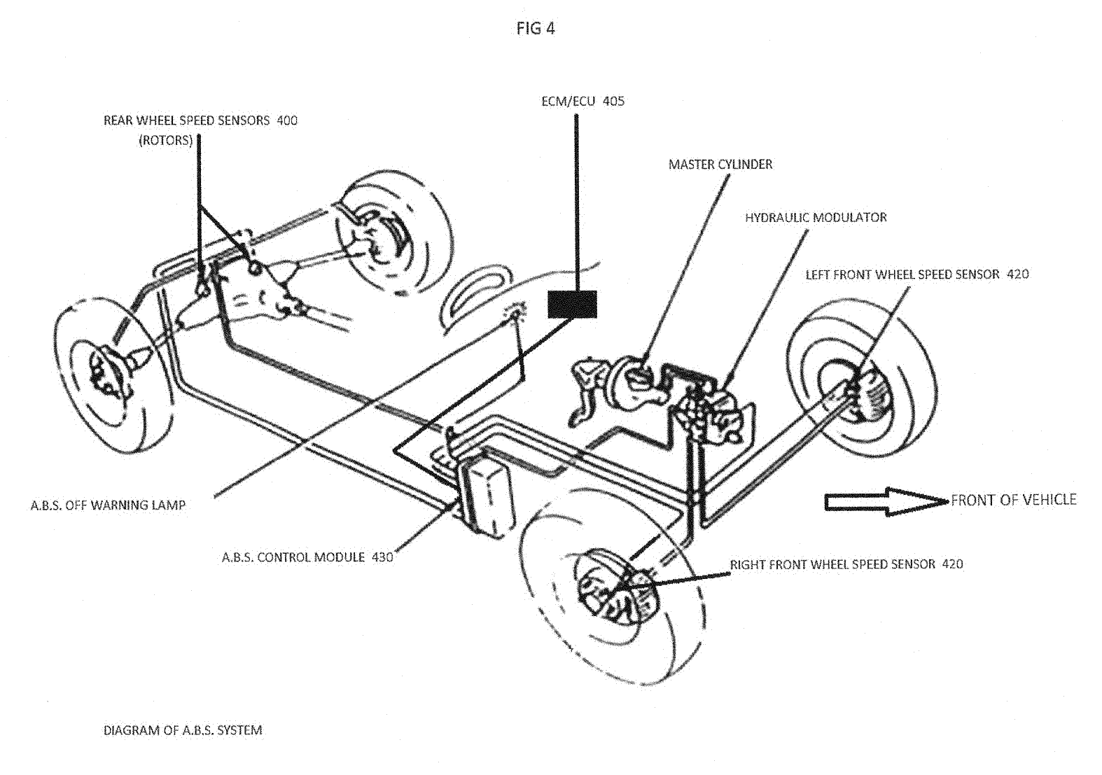

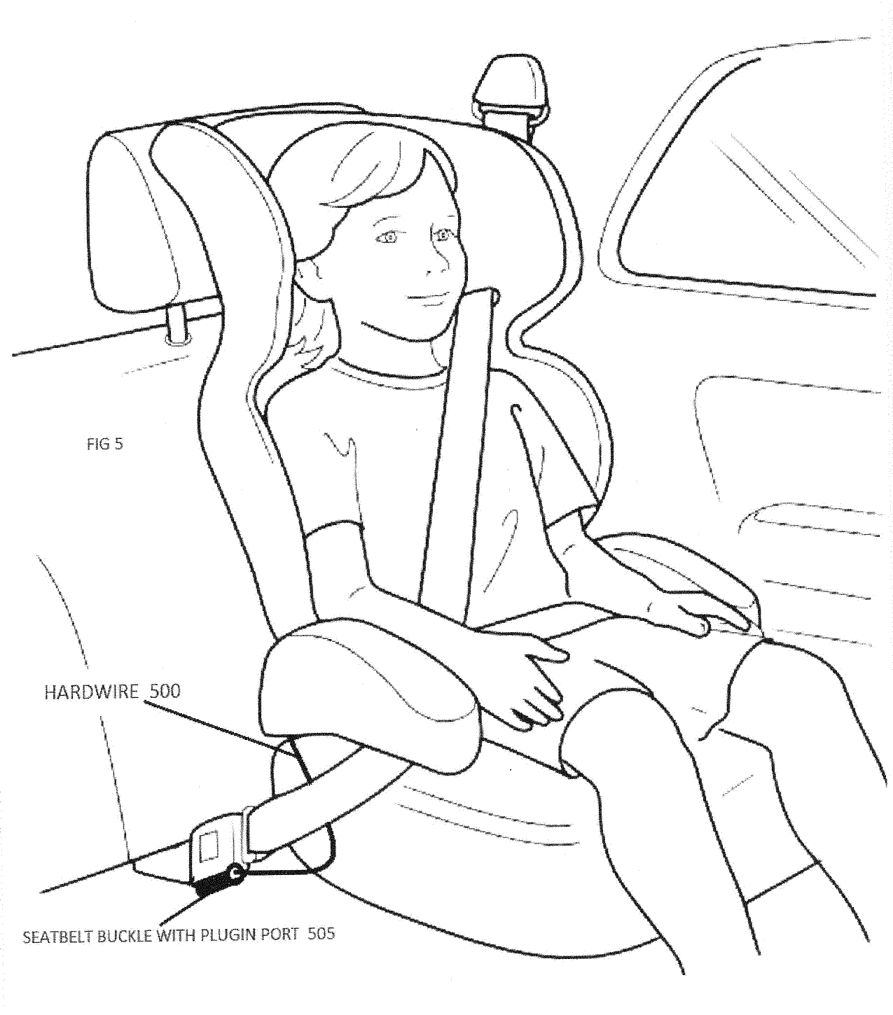

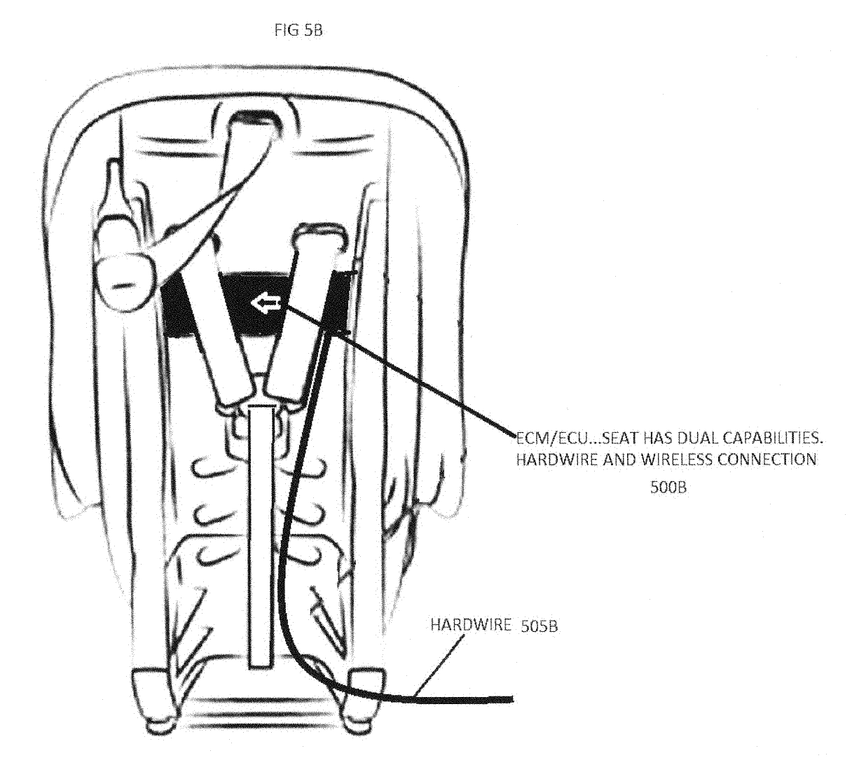

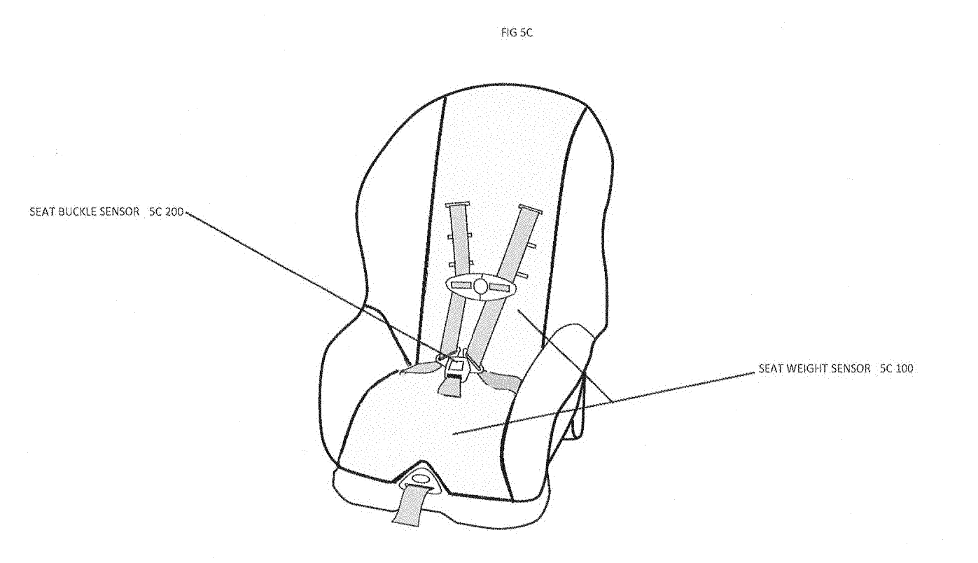

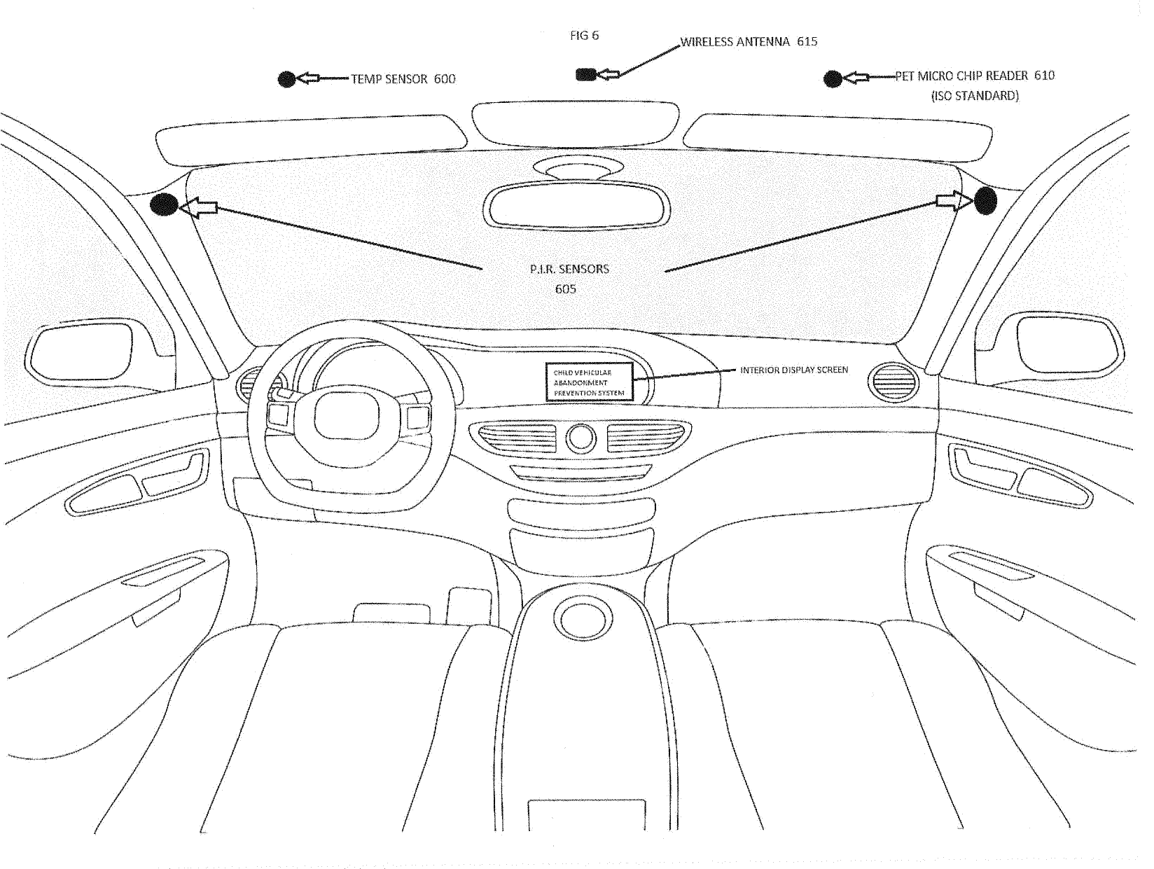

FIG. 4 depict a skeleton view of a ABS brake system. ABS is a safety system that prevents a vehicle from skidding or sliding when the brakes are applied. When the brake is applied, the ABS control module FIGS. 4/430 reads the speed data from the wheel speed sensors FIGS. 4/420 & 400 and sends the correct pressure to each wheel to prevent any sliding/skidding (wheels locking up). The present invention will use ABS wheel speed sensors FIGS. 4/420 & 400 to determine when the vehicle is in motion. The job of the wheel speed sensors FIGS. 4/420 & 400 is to constantly monitor and report the rotational speed of each tire to the ABS control module FIGS. 4/430. On vehicles that have rotors, each wheel will have a wheel speed sensor FIGS. 4/420. On vehicles that have rear drums (instead of rotors), one sensor would monitor both wheels. The present invention will read all wheel speed sensors FIGS. 4/420 & 400 on a vehicle via the ABS control module FIGS. 4/430. Vehicle motion detection stops all attempt to circumvent the system by placing pseudo weight on a weight sensored seat FIGS. 2/220. Vehicle motion detection will work in conjunction with the passive infrared sensors (P.I.R) FIGS. 6/605. If there is weight in a weight sensored seat FIGS. 2/220, but no vehicle motion, the passive infrared sensors FIGS. 6/605 would detect for human presence, if the passive infrared sensors FIGS. 6/605 doesn't detect a human, the system would immediately alarm in secure mode FIGS. 2/200, 280, 295, 230, 240, 235, 250 & 270. When a vehicle owner first approach a vehicle equipped with the Child Vehicular Abandonment Prevention System, upon the door ajar sensor FIGS. 2/226 being tripped, a signal would be sent to the ECM/ECU FIGS. 1, 1B & FIGS. 2/299 to wake the child seat FIGS. 5B, 5 & 5C. The child seat FIGS. 5B, 5 & 5C will remain awake until the ABS wheel speed sensor FIGS. 4/420 & 400 detects motion. If vehicle owner didn't place a child in the child seat FIGS. 5B, 5 & 5C, the ECM/ECU FIGS. 1, 1B & FIGS. 2/299 would place the child seat FIGS. 5B, 5 & 5C back into Sleep mode. A secondary method of vehicle motion detection would be the transmission speed sensor (TSS), also commonly referred to as a vehicle speed sensor (VSS). Vehicle motion detection is for stopping an attempt to circumvent the system by placing pseudo weight on a weight sensored seat FIGS. 2/220.

FIG. 5 depict the child seat with its hardwire FIGS. 5/500 plugged into the redesigned seatbelt buckle FIGS. 5/605. The hardwire FIGS. 5/500 connection leading from the child seat would plug directly into the port on the host vehicle redesigned seat belt buckle FIGS. 5/505 mechanism. Once connected, the ECM/ECU FIGS. 1, 1B & FIGS. 2/299 would then recognize the seat like a computer recognizes a thumb drive allowing verbal alerts/alarms to be relayed through the host vehicle speakers FIGS. 2/245 and the vehicle display screen FIGS. 7D/100. Interfacing between child seat FIGS. 5B, 5 & 5C and the ECM/ECU FIGS. 1, 1B & FIGS. 2/299 through the vehicle display screen FIGS. 7D/100 will allow the child seat FIGS. 68, 5 & 5C to be programmed to the child's name (such as the name of the child who will be occupying the seat) and displayed over the vehicle display screen FIGS. 7A/705A. Interfacing capability also allows the owner of the child seat FIGS. 5B, 5 & 5C to upload their information such as name and cell numbers to the child seat FIGS. 5B, 5 & 5C ECM/ECU FIGS. 5B/500B. With the child and owner information uploaded to the child seat ECM/ECU FIGS. 5B/500B, if the seat is removed from one caregiver vehicle to another caregiver vehicle equipped with the Child Vehicular Abandonment Prevention System, the child seat FIGS. 5B, 5 & 5C would sync to the new vehicle ECM/ECU FIGS. 1, 1B & FIGS. 2/299 along with the child's and owner's information by wireless FIGS. 2/220 or hardwire FIG. B connection. In this instance, if the new caregiver vehicle is triggered into secure mode FIGS. 2/200, 280, 295, 230, 240, 235, 260 & 270, the new caregiver vehicle ECM/ECU FIGS. 1, 1B & FIGS. 2/299 would text message the programmed seat owners from the new caregiver vehicle alerting the programmed owners of the situation. The child seat FIGS. 5B, 5 & 5C is capable of being programmed through USB by a home pc, laptop OF through the vehicle display screen FIGS. 7D/7D100 interfacing once sync FIGS. 2/220 or plugged in FIGS. 8/800. The child seat FIGS. 5C/100 will acknowledge a child by weight/presence, detecting 6 ounces and greater. If a parent or guardian place a child in a programmed child seat FIGS. 5B, 5 & 5C, said invention would say welcome to the child by their name over the vehicle interior speakers FIGS. 2/245. After saying welcome, said invention will repeatedly say the child's name over the vehicle speakers FIGS. 2/245 and that he/she is onboard (example; welcome Chris, Chris is onboard, Chris is onboard, Chris is onboard) over the vehicle speakers FIGS. 2/245 until the driver or someone occupies a weight sensored seat FIGS. 2/220. If a driver seat more than one child in programmed child seats FIGS. 5B, 5 & 5C, said invention would say welcome to each child as they are secured in their seats, with more than one child seated, the repeated interior speaker FIGS. 2/245 alert would say “children onboard”. If a child seatbelt FIGS. 5C/200 becomes unfasten, said invention would alert vocally over the interior speakers FIGS. 2/245 repeatedly saying the child's name and that the seatbelt FIGS. 5C/200 has become unfasten. If the child seat hardwire FIGS. 5/500 connection becomes disconnected from the seatbelt access port FIGS. 5/505, FIGS. 8/800 said invention wilt verbal alert over the vehicle speakers FIGS. 2/245 saying “wire connection has been lost”, said invention wireless antenna FIGS. 2/202 would then pick up child seat FIGS. 5B, 5 & 5C wireless signal. In both instances, the vehicle display screen FIG. 7D, would also display messages stating the loss of connectivity. Said invention would distinguish alerts/alarms by each child seat FIGS. 5B, 5 & 5C. When the driver has reached their destination, as soon the vehicle door ajar FIGS. 2/225 is detected, said invention will alert over the vehicle speakers FIGS. 2/245 repeatedly saying the child's name and that they are onboard (Chris is onboard, Chris is onboard, Chris is onboard/if more than one child, it would repeatedly say children are onboard) until the child/children are removed from the child seat FIGS. 5B, 5 & 5C. When a driver exits the vehicle removing weight from the seat weight sensor FIGS. 2/220, the ECM/ECU FIGS. 1, 1B & FIGS. 2/299 will allow 3 minutes for driver to remove child from the child seat FIG. 5. If child isn't removed in 3 minutes, system will alarm (vehicle alarm only) FIGS. 2/280 for 1 minute before going into secure mode FIGS. 2/200, 280, 295, 230, 240, 236, 299, & 270 believing the child has been abandon. The 1 minute is to alert the driver and or that time has expired before going into full secure mode FIGS. 2/200, 280, 295, 230, 240, 235, 250 & 270 which in addition to the vehicle alarm, the alarm voice module, power door locks locking, ignition starting vehicle, elevating windows, temp sensor determining A/C or heat, A/C or heat being turned on, text message a logged love one and dialing 911 and GPS location at 10 minutes of alarming. When not in use, child seat FIG. 6 will go into sleep mode. The child seat FIGS. 5B, 5 & 5C awakens by vehicle door ajar FIGS. 2/225 w/o ignition FIGS. 2/230 on or vehicle engine running. When a vehicle owner first approach a vehicle equipped with the Child Vehicular Abandonment Prevention System, upon the door ajar sensor FIGS. 2/226 being tripped, a signal would be sent to the ECM/ECU FIGS. 1, 1B & FIGS. 2/299 to wake the child seat FIGS. 5B, 5 & 5C. The child seat FIGS. 5B, 5 & 5C will remain awake until the ABS wheel speed sensor FIGS. 4/420 & 400 detects motion. If child seat FIGS. 5B, 5 & 5C weight/occupancy sensor FIG. 5C haven't detected a presence once wheel speed sensors FIGS. 4/420 & 400 detects motion, the ECM/ECU FIGS. 1, 1B & FIGS. 2/299 would place the child seat FIGS. 5B, 5 & 5C back into sleep mode. If a child or children are placed in the child seats 19G. 5B, 5 & 5C, the driver will have 3 minutes per child seat FIGS. 5B, 5 & 5C to secure each child and occupying a weighted sensor seat FIGS. 2/220 to detour secure mode alarming FIGS. 2/200, 280, 295, 230, 240, 235, 299 & 270. Said invention will not discriminate from wireless sync or plugged in child seats FIG. 5B to execute time limit. If only one child is being seated in a child seat FIGS. 5B, 5 & 5C rather sync or plugged into the system, the system will have a 3-minute limit for a driver to secure the child and occupy a weight sensored seat FIGS. 2/220. If 2 children are being seated, driver would have 6 minutes to occupy a weight sensor seat FIGS. 2/220; if 3 children are being seated, driver would have 9 minutes to seat all children and occupy a weight sensor seat FIGS. 2/220 to thwart secure mode alarming FIGS. 2/200, 280, 295, 230, 240, 235, 250 & 270. The ECM/ECU timer FIGS. 1/100 would reset the 3 minutes once the next child is placed in their child seat FIGS. 5B, 5 & 5C and the child seat FIGS. 5B, 5 & 5C detects weight/presence FIGS. 5C/100. When the driver has reached their destination with 2 or more children onboard and exit the vehicle, if the driver doesn't remove the first child in 3 minutes, system will alarm (vehicle alarm only) FIGS. 2/280 for 1 minute before going into secure mode FIGS. 2/200, 280, 295, 230, 240, 235, 250 & 270 believing the child has been abandon. The 1 minute is to alert the driver and or that time has expired before going into full secure mode FIGS. 2/200, 280, 295, 230, 240, 235, 250, & 270 which in addition to the vehicle alarm, the alarm voice module, power door locks locking, ignition starting vehicle, elevating windows, temp sensor determining A/C or heat, NC or heat being turned on, text message a logged love one and dialing 911 and GPS location at 10 minutes of alarming. If the 1 minute vehicle alarm FIGS. 2/280 is triggered, caregiver would still have an opportunity to remove child/children during alarming before vehicle goes into secure mode FIGS. 2/200, 280, 296, 230, 240, 235, 260, & 270 by removing child/children from their child seat which would cease the vehicle alarm FIGS. 2/280 and thwart secure mode FIGS. 2/200, 280, 295, 230, 240, 235, 250, & 270 alarming. The child seat ECM/ECU FIGS. 5B/500B would signal the ECM/ECU FIGS. 1, 1B & FIGS. 2/299 to restart the 3 minutes each time a child is removed from a child seat FIGS. 5B, 5 & 5C. The child seat ECM/ECU FIGS. 5B/500B would be mounted on rear of child seat FIGS. 5B, 5 & 5C. All above applies to infant carrier seats.

FIG. 6 depict a cockpit interior view of a vehicle displaying the position of the temp sensor 600, passive infrared sensors 605, ISO pet microchip reader 610 and the wireless antenna 615. The temp sensor FIGS. 6/600 will read the vehicle inside temperature only. The sole purpose of the temp sensor is determining rather the A/C or heat FIGS. 2/260 would be turned on during secure mode alarming FIGS. 2/200, 280, 295, 230, 240, 235, 250 & 270. The temp sensor FIGS. 6/600 is not used to trigger the system to alarm due to perilous interior temperature, but as said above, the temp sensor FIGS. 6/600 only determines rather the NC or heat FIGS. 2/250 will be turned on during secure mode alarming FIGS. 2/200, 280, 295, 230, 240, 235, 250 & 270. Said invention does not alarm by temperature threat, secure mode alarming FIGS. 2/200, 280, 295, 230, 240, 235, 260, & 270 is triggered after the vehicle alarm system has alarmed for 1 minute due to the 3-9 minute timer has been ignored and weight being removed from all weight censored seats FIGS. 2/220 with a child/children still seated in the child seat FIGS. 5B, 5 & 5C or a pet haven't been removed leaving the system to believe a child/children or pet has been left unattended. The vehicle alarm system FIGS. 2/280 would first alarm for 1 minute. The 1 minute is to alert the driver and or that time has expired before going into full secure mode FIGS. 2/200, 280, 295, 230, 240, 235, 250 & 270 which in addition to the vehicle alarm, the alarm voice module, power door locks locking, ignition starting vehicle, elevating windows, temp sensor determining A/C or heat, A/C or heat being turned on, text message a logged love one and dialing 911 and GPS location at 10 minutes of alarming. FIGS. 6/605 Represents passive infrared sensors. The term passive in this instance refers to the fact that PIR devices do not generate or radiate any energy for detection purposes. They work entirely by detecting the energy given off by other objects. PIR sensors don't detect or measure “heat”; instead they detect the infrared radiation emitted or reflected from an object. All objects with a temperature above absolute zero emit heat energy in the form of radiation. Usually this radiation isn't visible to the human eye because it radiates at infrared wavelengths, but it can be detected by electronic devices designed for such a purpose. Seat weight sensors FIGS. 2/220 detection can possibly be circumvented by placing weighted items on the seat. For this cause, said invention would have P.I.R (Passive Infrared Sensors) sensors FIGS. 6/605 capable of detecting the entire vehicle interior. The P.I.R. FIGS. 6/606 sensor will work in conjunction with the ABS wheel sensor FIGS. 4/400 & 420 for motion detection. Meaning, if there's no vehicle motion and a child is seated in the child seat FIGS. 5B, 5 & 5C and weight is present in a weight sensored seat FIGS. 2/220, the P.I.R. sensors FIGS. 6/605 would activate to make sure the weight is human not pseudo. If no presence is detected, said invention will immediately alarm in secure mode FIGS. 2/200, 280, 295, 230, 240, 235, 250 & 270 believing there is an attempt to circumvent the system and an abandonment situation. No motion consists of the transmission in any gear and engine running or not or parked. FIGS. 6/610 is a ISO pet microchip reader (International standards Organization), the global standard that is consistent worldwide and use the ISO standard frequency is 134.2 kHz. Since pet chips don't operate on the same frequency, the ISO capable readers are capable of reading all frequencies. For example, if a dog was implanted with an ISO standard microchip in the U.S. travels to Europe with its owners and becomes lost, the ISO standard scanners in Europe would be able to read the dog's microchip. If the dog was implanted with a non-ISO microchip and the ISO scanner was not forward- and backward-reading (universal), the dog's microchip might not be detected or be read by the scanner. The ISO chip reader FIGS. 6/610 is for detecting pets in a vehicle and would prompt the system to acknowledge a pet once it enters the vehicle by saying welcome to the pet by its name if logged. If a microchipped pet was left in a vehicle equipped with said invention, the vehicle would alarm in secure mode FIGS. 2/200, 280, 295, 230, 240, 235, 250, & 270 announcing “pet onboard unattended”. If a child and pet is left in a vehicle equipped with said invention, vehicle would alarm in secure mode FIGS. 2/200, 280, 295, 230, 240, 235, 250, & 270 announcing the threat to the child, “child onboard unattended”. If a pet is in the vehicle, as soon as the system detects the vehicle door ajar FIGS. 2/225, it would alarm over the vehicles speakers FIGS. 2/245 saying pet onboard or by the pet's name if it has been programmed into the system saying the pet's name is onboard. If a child and pet was in the vehicle, as soon as the system detects the vehicle door ajar FIGS. 2/225, it would alarm over the vehicles speakers FIGS. 2/245 saying child name is onboard if togged. Said invention will continue to alarm until pet is removed from the vehicle. Driver will have 3 minutes to remove pet from the vehicle after exiting from weight sensored seat FIGS. 2/220 of vehicle. If pet isn't removed in 3 minutes and no one is occupying a weight sensored seat FIGS. 2/220, system will alarm (vehicle alarm only) FIGS. 2/280 for 1 minute before going into secure mode FIGS. 2/200, 280, 296, 230, 240, 236, 250, & 270 believing the pet has been abandon. The 1 minute is to alert the driver and or that time has expired before going into full secure mode FIGS. 2/200, 280, 295, 230, 240, 235, 250, & 270 which in addition to the vehicle alarm, the alarm voice module, power door locks locking, ignition starting vehicle, elevating windows, temp sensor determining NC or heat, NC or heat being turned on, text message a logged love one and dialing 911 and GPS location at 10 minutes of alarming. FIGS. 6/615 is a wireless antenna to accommodate the wireless capability of the child seat FIG. 5B. The wireless capability will transmit wireless communication between the ECM/ECU FIGS. 1, 1B & FIGS. 2/299, the child seat FIGS. 5B, 5 & 5C & for the interior display screen FIGS. 7D/7D100 to display TempTraq (US 20140121557 A1) 24-hour intelligent thermometer that's wearable, wireless and continuously monitors a child's temperature to be displayed on the vehicle's interior display screen FIGS. 7/700.

FIGS. 7, 7A, 7B, 7C & 7D depict the interior display of a vehicle. It will allow access to said invention functions such as refueling mode FIG. 7C, live display of a child's temperature FIG. 7 through TempTraq (US 20140121557 A1) 24-hour intelligent thermometer that's wearable, wireless and continuously monitors a child's temperature. Programming through touch screen capability and display alarms/alerts situation status. Vehicle owners would have interface interaction with the system through the vehicle display screen FIG. 7D. Pictures of a child or pet would be displayed through the display screen when alarming/alerting, welcoming a child or pet and special events such as birthdays FIGS. 7A/705A. FIGS. 7B/710B will display If vehicle goes into secure mode FIGS. 2/200, 280, 295, 230, 240, 235, 250, & 270.

FIG. 8 depict a conventional seatbelt buckle redesigned with an access port to allow FIGS. 5/500 & FIGS. 5B/505B to plug into the seatbelt buckle FIG. 8. Every vehicle rear seat buckle FIG. 8 will have an access port FIGS. 8/800 without losing integrity to secure occupants. The hardwired harness FIGS. B/B1 bridges the connection between the ECM/ECU FIGS. 1, 1B & FIGS. 2/299 and the seatbelt access port FIGS. 8/800 from underneath the vehicle seating while FIGS. 5/500 & FIGS. 6B/605B will connect to the seatbelt access port FIGS. 5/505.

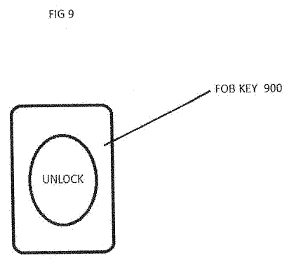

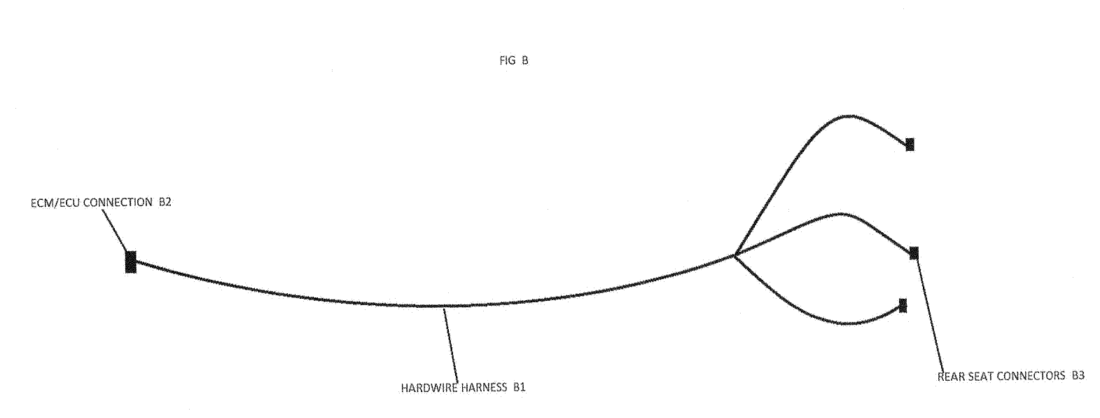

FIGS. 9/900 depict a key fob with only an unlock feature. It is a keyless entry device that only first responders would have to disarm/unlock a Child Vehicular Abandonment Prevention System equipped vehicle when in secure mode FIGS. 2/200, 280, 295, 230, 240, 236, 260, & 270. First responders typically include Police Officers, Deputy Sheriffs, Troopers, Firefighters, Paramedics, EMT and Rescuers. Key fob FIGS. 9/900 may also be given to any CERTIFIED Citizens Patrol program. Civilians who may investigate an alarming Child Vehicular Abandonment Prevention System equipped vehicle wouldn't have to attempt to gain access inside the vehicle due to the system would make it comfortable for the child (AC or heat FIGS. 2/250) and secure (doors locked FIGS. 2/240). In most cases, civilians who witness an alarming Child Vehicular Abandonment Prevention System equipped vehicle, would call authorities before the 10 minutes' lapse at which said invention would phone authorities, this eliminates premature calls to authorities from said invention. A first responder would come and unlock the vehicle due to the vehicle entering secure mode FIGS. 2/200, 200, 295, 230, 240, 236, 250, & 270 which denotes a possible abandonment situation. This gives authorities the opportunity to question the caregiver and investigate the situation. If system is provoked to alarm in secure mode FIGS. 2/200, 280; 296, 230, 240, 235, 250, & 270, but a door or doors are left open, said invention would trigger an abridged version of full secure mode FIGS. 2/200, 280, 295, 230, 240, 235, 250, & 270 in which of FIGS. 2/200, 280, 295, 230 would only activate and the ECM/ECU FIGS. 1, 1B & FIGS. 2/299 would “immediately” contact 911 FIGS. 2/295, text message logged love ones, give gps location and lock the ignition FIGS. 2/230 preventing anyone starting the vehicle and wouldn't start the vehicle if doors are open. FIGS. 9/900 is only capable of triggering the ECM/ECU FIGS. 1, 1B & FIGS. 2/299 to disarm/unlock a Child Vehicular Abandonment Prevention System equipped vehicle that is in secure mode FIGS. 2/200, 280, 295, 230, 240, 235, 250, & 270; FIGS. 2/230, 200, 280 & 295. Once a Child Vehicular Abandonment Prevention System equipped vehicle is triggered into secure mode FIGS. 2/200, 280, 295, 230, 240, 235, 299, & 270 the vehicle owner hard key or key fob wouldn't be capable of unlocking the vehicle. Secure mode FIGS. 2/200, 280, 295, 230, 240, 235, 250, & 270 would block the hard key from unlocking the vehicle doors and cancel out the owner's key fob signal until the vehicle has been disarmed/unlocked from secure mode FIGS. 2/200, 280, 296, 230, 240, 236, 250, & 270 by key fob FIGS. 9/900. Key fob FIGS. 9/900 would use a different signal from the vehicle's keyless entry signal to trigger the ECM/ECU FIGS. 1, 1B & FIGS. 2/299 to disarm/unlock. The vehicle hard key door lock will not allow the hard key to unlock the vehicle door when in secure mood.

FIG. B depict a hardwire harness with a ECM/ECU FIGS. 1, 1B & FIGS. 2/299 connection FIGS. B2 and 3 rear seatbelt buckles connections FIG. B3. The hardwire harness FIG. B1 would be mounted in vehicles at production. Its bridges the ECM/ECU FIGS. 1, 1B & FIGS. 2/299 and child seat FIGS. 5, 5B & 5C. It's purpose is to transmit and receive electronic messages, alerts and alarms from the ECM/ECU FIGS. 1, 1B & FIGS. 2/299 & the child seat FIGS. 5, 5B & 5C. The hardwire harness FIG. B would also maintain a charge and recharge the child seat FIGS. 5, 5B & 5C.

What is claimed is:

1. A Child Vehicle Abandonment Prevention System method seeking to totally defunct heat stroke deaths and abandonment of children and pets in vehicles rather mistakenly or intentionally while utilizing native vehicle components and several non-native vehicle components comprising: an ecm/ecu equipped with the latest android technology, timer, abs wheel speed sensors/transmission vss, vehicle interior speakers, power windows, flow sensor/meter, ultra-sonic flow meter/sensor, interior display screen, iso microchip reader, a/c & heat, ignition, an alarm voice module, vehicle door ajar sensor, vehicle seat weight sensors, power door locks, a hardwire harness, temperature sensor, vehicle alarm system, wireless antenna transmit/receive, passive infrared sensors, redesigned seatbelt buckle with an hardwire access port, 911 call capability and gps location, text messaging, wireless antenna and fob key; and a child seat with an ecm/ecu, seat weight sensor, seat buckle sensor and hardwire.

2. The method of claim 1 wherein all the following constituents will nexus to the ecm/ecu by harness or wirelessly; abs wheel speed sensors/transmission vss, vehicle interior speakers, power windows, flow sensor/meter, ultra-sonic flow meter/sensor, interior display screen, pet iso microchip reader, a/c & heat, ignition, an alarm voice module, vehicle door ajar sensor, vehicle seat weight sensors, temperature sensor, vehicle alarm system, passive infrared sensors and power door locks, wireless antenna.

3. The method of claim 1 further comprising the constituents signaling the ecm/ecu through the harness nexus or wirelessly wherein ecm/ecu receives the signals through the harness nexus or wirelessly.

4. The method of claim 1 further comprising the ecm/ecu signaling the constituents through the harness nexus or wirelessly wherein the constituents receives the signals through the harness or wirelessly.

5. The method of claim 2 wherein the constituents; abs wheel speed sensors and transmission vss nexus to the ecm/ecu via the abs control module wherein the flow meter sensor is situated in the fuel filler neck and nexus to the ecm/ecu wherein the ultra-flow meter sensor is situated in the fuel filler neck and nexus to the ecm/ecu wherein the pet iso microchip reader is mounted on the ceiling of the vehicle and nexus to the ecm/ecu wherein the vehicle interior speakers nexus to the ecm/ecu wherein the interior display screen nexus to the ecm/ecu wherein the vehicle door ajar sensor nexus to the ecm/ecu wherein the seat weight sensors nexus to the ecm/ecu wherein the wireless antenna is mounted on the ceiling and receives and transmits to the ecm/ecu wherein the passive infrared sensors are mounted on the roof support structure and nexus the ecm/ecu wherein the ignition nexus to the ecm/ecu wherein the ac & heat nexus the ecm/ecu wherein the alarm voice module nexus the ecm/ecu wherein the temperature sensor mounted on the ceiling of the vehicle nexus the ecm/ecu wherein the vehicle alarm system nexus the ecm/ecu wherein the power windows nexus the ecm/ecu.

6. The method of claim 3 wherein the hardwired harness nexus the ecm/ecu and child seat via the redesigned seatbelt buckles hardwire access ports.

7. The method of claim 4 wherein the timer manages the loading and unloading of children and pets, the 1 minute alert before going into secure mood and the exiting and reentry time of caregiver/driver.

8. The method of claim 5 wherein the child seat awakes wherein detects presence/occupancy wherein seat belt sensor continuously monitors wherein a child's temperature would be displayed live over the vehicle display screen; also applies to infant carriers.

9. The method of claim 6 wherein the following features comprise “SECURE MODE” and is simultaneously activated by the ecm/ecu; power door locks locking, power windows elevating, temperature sensor determining ac or heat, ignition starting, alarm voice module verbal message, vehicle alarm system, text messaging logged caregiver wherein 911 call and gps location activates at 10 minutes of alarming.

10. The method of claim 7 further comprising an abridged method of “SECURE MODE”; ignition locking, alarm voice module verbal message, vehicle alarm system, text messaging logged caregiver; immediate 911 call and gps location.

11. The method of claim 8 wherein the fob key disarms/unlocks the ecm/ecu from secure mode.
